(19) United States
(12) Patent Application Publication
(10) Pub. No.: US 2008/0051744 A1
(43) Pub. Date: Feb. 28, 2008

(54) Sanitary/hygienic products with individual disposal system

(76) Inventor: Thomas F. Cummings, Westport, CT (US)

Correspondence Address: SOFER & HAROUN LLP., 317 MADISON AVENUE, SUITE 910, NEW YORK, NY, 10017, US

(21) Appl. No.: 11/799,748

(22) Filed: May 2, 2007

Related U.S. Application Data
(60) Provisional application No. 60/799,414, filed on May 10, 2006.

(51) Int. Cl. A61F 13/15

(52) U.S. Cl. 604/359; 604/385.13

(57) ABSTRACT

A disposal system for sanitary products is provided comprising a disposable sanitary product; a pocket attached to the sanitary product, the pocket having an opening; a disposal bag having a cavity and opening means communicating with the cavity. The bag is formed into a configuration of reduced dimensions and is located and retained in the pocket. The bag is accessible through the opening of the pocket, whereby the bag may be extended to enlarged dimensions after use of the sanitary product. The opening means comprises a short tag attached to the mouth of the bag, protruding from the opening of the pocket; and a long tag having a first end and a second end, the first end attached to the mouth of the bag and the second end attached to the sanitary product within the pocket, so that when a user pulls on the short tag the bag is released from the pocket yet remains attached to the sanitary product by the second end of the long tag. As the user continues to pull on the short tag, the mouth of the bag is opened to enable placement and protection of one hand inside the bag to then grasp the soiled sanitary product from other hand, and subsequently enable inverting the bag over the soiled sanitary product and retention therein.

### RELATED APPLICATIONS

The present application claims priority to Provisional Application No. 60/799,414 filed on May 10, 2006, for a Diaper Pocket with Disposable System, and is hereby incorporated by reference.

### FIELD OF INVENTION

The invention relates to the field of disposable sanitary products, more particularly to a system for efficiently disposing used sanitary products.

### BACKGROUND

Currently, one cannot conveniently change and readily make a proper disposal of used sanitary/hygienic products (e.g. adult incontinence pads and underwear, protective briefs, under-pads for adults to sit/lay on, adult diapers, sanitary napkins, pads and/or shields, diapers, etc.). After being soiled, disposable sanitary products are usually folded and secured by the reusable adhesive fasteners. However, after changing a soiled sanitary product, one must still worry about the risk of waste leaking on surrounding objects between the changing area and the disposal receptacle. In this process, one needs to prepare in advance, use both hands, switch hands, and/or grope around for some kind of wrapper, or bag, or frequently lay the product down and eventually dispose of the used product inconveniently in awkward places, even in an unsanitary manner. Especially when traveling, visiting with other people, at their homes, in restaurants, movies, place of work, the mall, at the beach, etc., this situation can be very embarrassing and certainly does not facilitate hygienic disposal.

Nothing in the prior art has delineated a complete system that is actually simple, efficient, and above all a really practical solution. The fact that such products say, “Dispose of in a sanitary manner,” and “Don't flush,” and/or “Put in some receptacle” is actual evidence that the basic need and problem has not been addressed. The current changing and disposal process is inefficient, inconvenient, messy, and unsanitary.

Various attempts have been made to design a disposable containment system however the disposal problem of sanitary products has not been completely overcome. U.S. Patent Application Publication No. 2005/0256487 to Williams discloses a disposal bag folded to fit neatly within a pouch on the rear portion of a diaper. A user reaches into the opening of the pouch, grasps and withdraws the disposal bag and unfolds the bag. The user then envelopes the soiled disposable article in the bag by reaching into the open end and grasping the article using the wall of the bag. User then inverts the bag around the article to contain it. The bag can then be tied and sealed.

U.S. Pat. No. 5,037,414 to Booth discloses a containment pouch which is removable from a pocket along the back marginal edge of a diaper through a ruptured opening. The containment pouch is folded in a stack of accordion pleats. After the pouch has been removed through the ruptured opening, the pouch is opened and the rolled up diaper is inserted.

U.S. Pat. No. 4,931,052 to Feldman discloses a storage container means mounted to the outer surface of the diaper which includes a pocket that is expandable to form a disposal container for the soiled diaper. The storage container means may be employed as a diaper disposal system separate and apart from the diaper and thus may be sold individually. Pockets sold in this manner will contain a suitable adhesive backing for attaching to the diaper.

U.S. Patent Application No. 2002/0133134 to Wilbon discloses a detachable sack with a layer of clear plastic covering the folded sack to keep it attached to the diaper. The detachable sack can be kept inside small areas apart from the diaper before and after use without requiring a lot of storage space.

U.S. Pat. No. 4,430,087 to Azpiri discloses a tubular storage bag having a closed end and an open end stored in a folded condition between the liquid impervious backing sheet and the absorbent sheet of the diaper. When the diaper has become soiled, access to the bag is gained by separating the backing sheet from the absorbent sheet. Bag is then deployed by manually unfolding from the interior of the diaper.

U.S. Pat. No. 4,743,240 to Powell discloses a bag formed integrally with the diaper so that a soiled diaper can be stored within the bag by placing one's hand within the bag, grasping the diaper and finally pulling the soiled diaper within the bag by inverting the bag.

U.S. Patent Application Publication No. 2005/0267432 to Sundberg et al. discloses a double action backing strip which is formed by a first part attached to the disposable pad, forming a storage pouch holding a disposal bag, and a second part separately attached to the disposable pad able to hold other hygienic products. The storage pouch is opened by pulling on the backing strip to first open the second part and release the further hygienic products and by further pulling, the bag structure is able to enclose the used disposable pad for hygienic disposal. This allows one handed operation and activates the bag without separate handling.

U.S. Pat. No. 5,702,379 to Preiss discloses a bag folded up and releasably attached to the outer layer of the diaper. The bag encloses cleaning items before the package is opened or used and thereafter can be used as a disposal bag for a soiled diaper.

U.S. Pat. No. 3,927,674 to Scharr discloses a diaper with a disposable bag located in the waistline portion. The bag may be removed from the diaper after which it may be unfolded. The soiled diaper may then be inserted through the opening means into the cavity of the bag. Alternatively, the user may place his hand in the cavity of the unfolded bag to grasp the soiled diaper through the bag, after which the bag is inverted to cover the soiled diaper.

### SUMMARY OF INVENTION

The object of the present invention is to provide a disposal system that is practical and efficient. The disposal system of the present invention can be an inherent part of standard adult sanitary/hygienic products, or it can be an individual add-on component allowing a user to integrate such a system with these products at his discretion. Furthermore, the disposal system can be employed for use with children's diapers. The present invention enables a user to easily, conveniently, quickly, and cleanly perform a simple efficient procedure for immediate and sanitary disposal of each used individual sanitary/hygienic product. The present invention can be conveniently used for almost all types of sanitary/hygienic products. The present invention enables each sanitary product and its disposal system to be integrated as one unit so that one does not have to carry around or locate separate bags. The bag within the pocket is so thin and specially folded such that it is unobtrusive and causes no discomfort.

Another advantage of the present invention is that it enables the user to, without switching hands, pull the bag out of the pocket, open the bag mouth, and insert one hand inside the bag. The system of the present invention also allows the user to naturally coordinate the use of two separate hands throughout the entire operation without having to lay the product down.

The present invention contains unique tabs of unique lengths having unique attachments which facilitate easy, clean, and quick pulling of the bag out of the pocket. One short tag is easily accessible to initially pull and that tag is attached to only one side of the bag's mouth, which also enables the simultaneous opening of the mouth of the bag as it is being pulled out. The system also facilitates easy, quick, and clean opening of the bag without letting go. A long tag is attached on one side to the opposite side of the bag's mouth and on its other side to the sanitary product inside of the pocket, in order to continuously create the necessary tension that simultaneously opens the bag as it is pulled out.

### BRIEF DESCRIPTION OF DRAWINGS

In order that the invention can be more readily understood, various embodiments will be described by way of illustration only with reference to the drawings wherein:

FIG. 1 is a plan view of the disposable sanitary product and pocket;

FIG. 2 is a perspective view of the add-on embodiment of the disposal system of the present invention;

FIG. 3 is a perspective view of the bag pulled out and opened from the disposal system of the present invention;

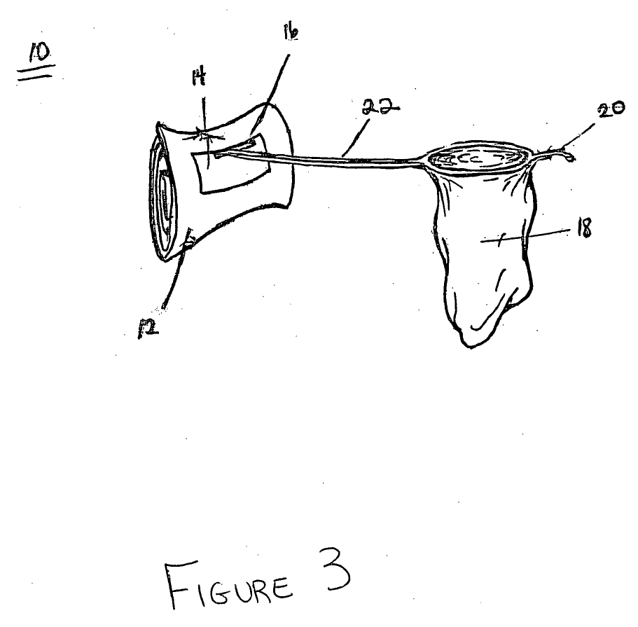

FIG. 4 is a fragmentary perspective view of the bag of the disposal system of the present invention after having been laterally folded into its reduced configuration according to the present invention;

FIG. 5 is a fragmentary perspective view illustrating the attachment means of the add-on embodiment of FIG. 2;

FIG. 6 is a fragmentary perspective view illustrating the protective covering for the attachment means of FIG. 5;

FIG. 7 is a fragmentary perspective view illustrating the corner of the add-on embodiment of FIG. 2 without the attachment means of FIG. 5; and

FIG. 8 is a flow chart illustrating a method of disposing sanitary products according to one embodiment of the present invention.

### DETAILED DESCRIPTION

The present invention has at least two alternative embodiments; an inherent standard part of a sanitary/hygienic product, or a separate add-on unit. The add-on alternative has a peel off back to expose adhesive which is then stuck onto a sanitary/hygienic type product, in advance or when needed or desired. This embodiment is directed more towards adult type products, unless it includes a feature to render the bag harmless if accidentally pulled out of a child's diaper.

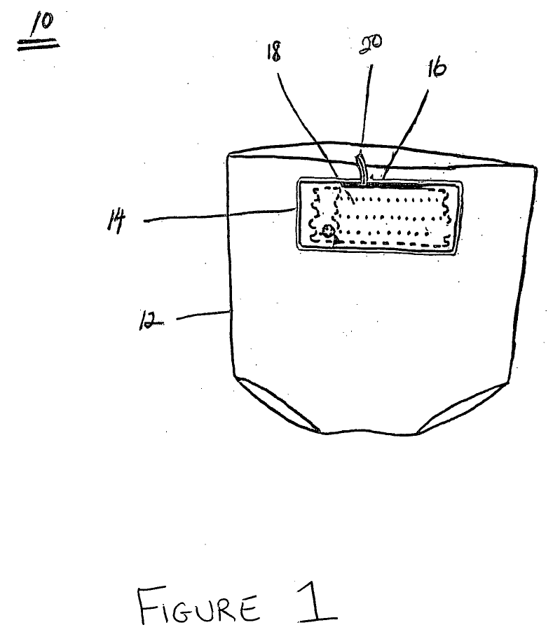

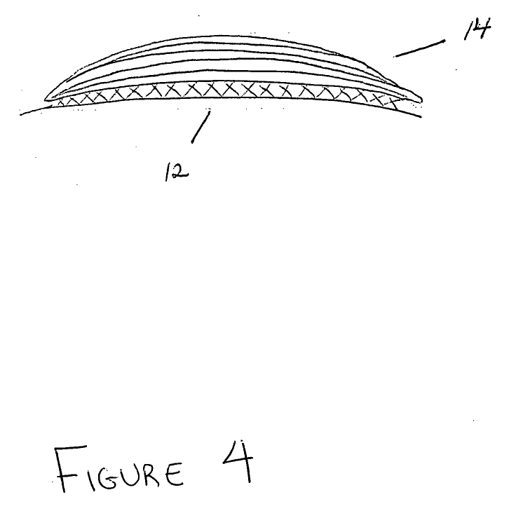

Referring to FIG. 1, one embodiment of the present invention is depicted. A sanitary product 12 has a small pocket 14 on one side. Pocket 14 contains a thin folded water-proof plastic bag 18 that is not obtrusive or uncomfortable. FIG. 4 illustrates bag 18 after having been laterally folded into its reduced configuration, so that it fits comfortably within pocket 14. In one embodiment of the present invention, bag 18 has at least one longitudinal fold prior to being folded into its reduced configuration. In another embodiment of the present invention, bag 18 is folded in accordion style. Pocket 14 has an opening 16 that is smaller than folded bag 18, so that bag 18 will not come out unless it is pulled through opening 16. In one embodiment, bag 18 is made of very thin plastic, approximately 10″×14″ so that it can be readily pulled out of pocket 14 through the opening. Bag 18 is also flexible, so that it may be inverted during placement of soiled sanitary product 12 into bag 18.

Bag 18 has two tags, a short tag 20, and a long tag 22, attached to opposite sides of bag's 18 mouth for the specific purpose of getting bag 18 out of pocket 14 and thereafter creating a natural tension between opposing forces by the two hands pulling tags 20, 22 in opposite directions which opens bag's 18 mouth. Short tag 20 is attached to the mouth of bag 18 and is easily accessible, protruding from opening 16 for the user to pull. In one embodiment, short tag 20 is approximately 1″ long. Long tag 22 is attached on one side to sanitary product 12 within pocket 14 and on its other side to the mouth of bag 18. The length of long tag 22 is longer than the depth of bag 18, so that the hands can be easily switched and bag 18 can then be easily inverted and pulled back down over used sanitary product 12. In one embodiment, the length of long tag 22 is approximately 15″ and is longer by approximately 1″ than the depth of bag 18.

By having bag 18 already open and at the end of long tag 22, a user can easily, quickly, and cleanly insert one hand inside bag 18, while the other hand is able to naturally continue to create the necessary tension to keep bag 18 in an open position. When inserted inside bag 18, the user's hand is covered so that it is protected in order to cleanly grasp used sanitary product 12.

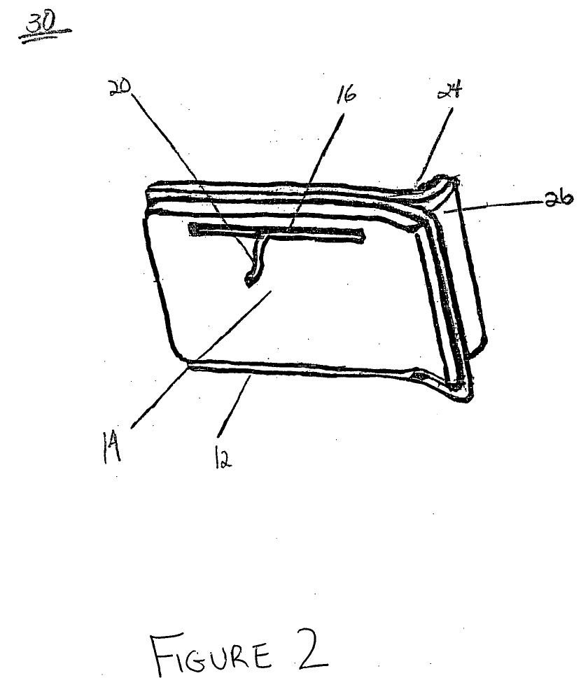

Referring to FIG. 2, the add-on embodiment 30 is illustrated. Add-on unit 30 comprises a pocket 14 having an opening 16. Pocket 14 contains a disposal bag 18 which is formed into a configuration of reduced dimensions. A short tag 20 is attached to the mouth of bag 18 and protrudes from opening 16 of pocket 14. A long tag 22 has a first end attached to the mouth of bag 18 and a second end attached to sanitary product 12 within pocket 14, so that when a user pulls on short tag 20, bag 18 is released from pocket 14, yet remains attached to sanitary product 12 by the second end of long tag 22. As the user continues to pull on short tag 20, the mouth of bag 18 is opened for placement of the soiled sanitary product 12 in bag 18.

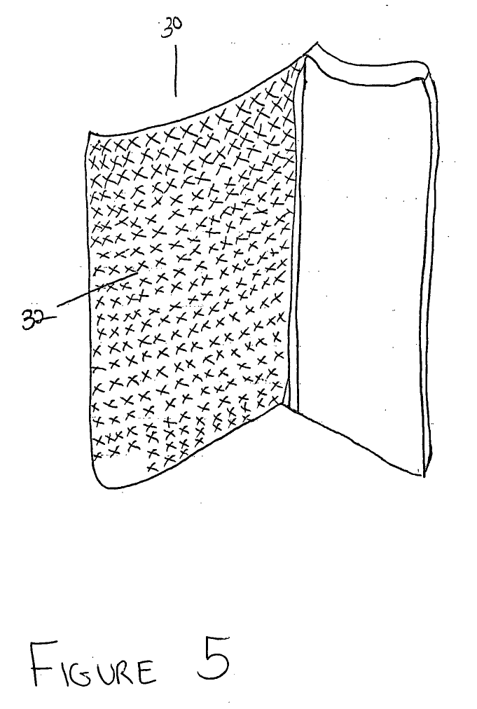

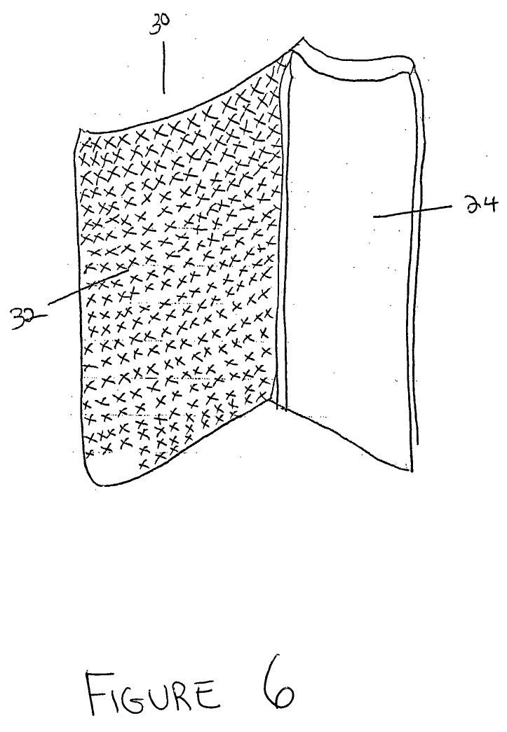

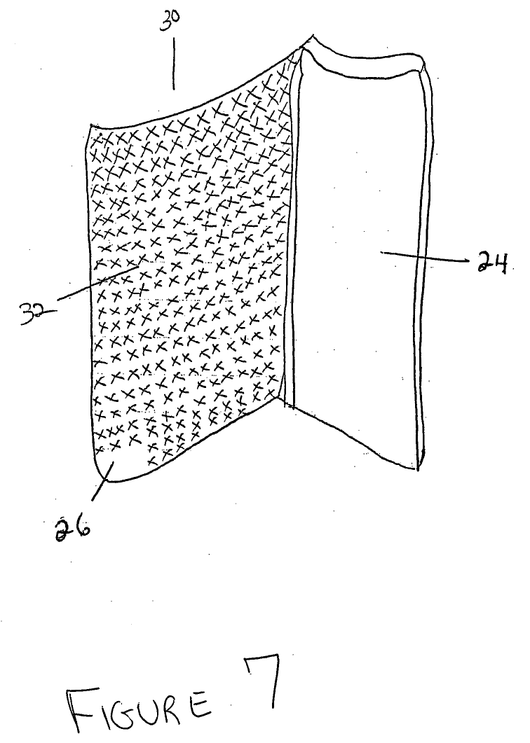

Add on unit 30 is stuck onto sanitary product 12 by placing the attachment means 32 which lines the outer surface of unit 30 in contact with product 12. FIGS. 5, 6, and 7 illustrate the attachment mechanism for add-on unit 30. Attachment means 32 which creates the bond between unit 30 and sanitary product 12 can be of any type of adhesive or bonding material. In one embodiment of the present invention, attachment means 32 is a quick release contact adhesive. Unit 30 also contains a removable protective backing 24 which is placed on top of attachment means 32, so that attachment means 32 of unit 30 only sticks to the desired location upon sanitary product 12 and is blocked from sticking to anything else. In this way, attachment means 32 is protected from diminished adhesive capability, since protective covering 24 prevents the adhesive from sticking to anything which it contacts. Unit 30 also has a corner 26 on its outer surface which lacks attachment means 32, so that protective backing 24 can easily be peeled away from unit 30 to then expose the attachment means 32 of unit 30 in order to stick unit 30 to sanitary product 12.

Further embodiments of the present invention could include a pleasing scenting and/or other items in pocket 14. If necessary, outside pocket 14 could have a textured, rougher surface for more friction. Another embodiment has a relatively larger opening with overlapping edges, with the overlay side up so that the bag could not come apart until pulled. For the embodiment on a baby diaper, a special tag may be attached to the inside of the pocket so that if the bag is pulled out of the child's diaper, it would tear a safety rip in the bag. In the alternative, a dual tag could be employed that unless separated before pulling, it would tear the bag when pulled out.

An alternative manufacturing technique for one embodiment of the present invention in order to minimize the number of separate pieces of the disposal system, bag 18, tags 20, 22 and pocket 14 can all be made out of one piece of continuous material.

Furthermore, the add-on embodiment approach of FIG. 2 could also serve as a way to manufacture a standard product. The standard product could be manufactured by laying a patch which is bigger than the folded bag over the bag, so that the edges of the patch, except for the slit, can be affixed to the sanitary product. In this embodiment, the sanitary product itself essentially becomes one side of the pocket. In yet another embodiment, rather than having a pocket affixed to the surface of the sanitary product, the pocket is manufactured and affixed inside the product, with a small slit in the product's surface, so the bag can be pulled through.

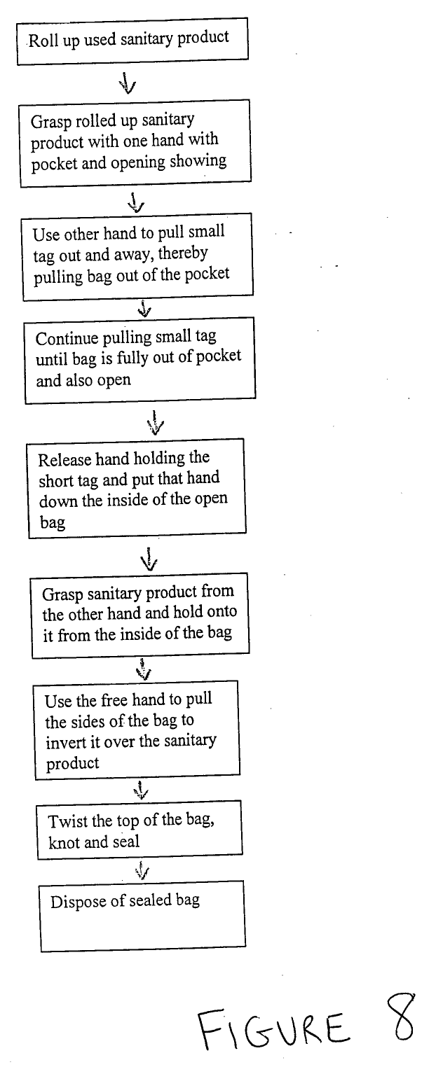

Referring to FIG. 8, a mode of operation is described according to one embodiment of the present invention. The soiled sanitary product 12 is removed and rolled up normally, in the direction that the side opposite pocket 14 is rolled up first and therefore ends up inside the roll with pocket 14 and opening 16 on the outside of the roll, exposed. One hand gently and firmly holds rolled product 12. The other hand grasps exposed short tag 20 and gently pulls bag 18 out of pocket 14, such that bag 18 is finally out of pocket 14 hanging down between two tags 20, 22. By utilizing the force of the two hands pulling in opposite directions, bag 18 is pulled open, without even releasing the hands. The hand that has been pulling bag 18 out and open is put inside open bag 18 to grasp used product 12. The now free hand that had been holding product 12 is now used to pull on tags 20, 22 and bag 18 in order to pull down and invert bag 18 over used product 12 which is now being held by the other hand from outside protective bag 18. Then both hands are free to twist, seal, and dispose of sanitary product 12.

Although the invention has been described with reference to specific embodiments, the foregoing description is not intended to be construed in a limiting sense. Various modifications to the disclosed embodiments as well as alternative applications of the invention will be suggested to persons skilled in the art. It is therefore contemplated that the appended claims will cover any such modifications, applications, or embodiments as fall within the true scope of the invention.

1. A disposal system for sanitary products comprising:
a disposable sanitary product;
a pocket attached to said sanitary product, said pocket having an opening;
a disposal bag having a cavity and opening means communicating with said cavity, said bag being formed into a configuration of reduced dimensions and being located and retained in said pocket, said bag being accessible through said opening of said pocket, whereby said bag may be extended to enlarged dimensions after use of said sanitary product, said opening means comprising a short tag attached to the mouth of said bag, protruding from said opening of said pocket; and a long tag having a first end and a second end, said first end attached to the mouth of said bag and said second end attached to said sanitary product within said pocket, so that when a user pulls on said short tag said bag is released from said pocket yet remains attached to said sanitary product by said second end of said long tag and as the user continues to pull on said short tag, the mouth of said bag is opened to enable placement and protection of one hand inside said bag to then grasp said soiled sanitary product from other hand, and subsequently enable inverting said bag over said soiled sanitary product and retention therein.

2. An add-on unit for the disposal of sanitary products comprising:
a pocket having an opening;
a disposal bag having a cavity and opening means communicating with said cavity, said bag being formed into a configuration of reduced dimensions and being located and retained in said pocket, said bag being accessible through said opening of said pocket, whereby said bag may be extended to enlarged dimensions after use of said sanitary product, said opening means comprising a short tag attached to the mouth of said bag, protruding from said opening of said pocket; and a long tag having a first end and a second end, said first end attached to the mouth of said bag and said second end attached to said sanitary product within said pocket, so that when a user pulls on said short tag said bag is released from said pocket yet remains attached to said sanitary product by said second end of said long tag and as the user continues to pull on said short tag, the mouth of said bag is opened to enable placement and protection of one hand inside said bag to then grasp said soiled sanitary product from other hand, and subsequently enable inverting said bag over said soiled sanitary product and retention therein; and
an attachment means on the outer surface of said pocket, so that said pocket can be adhered to a sanitary product at the user's discretion.

3. The disposal system for sanitary products of claim 1 wherein said pocket is a separate unit from said sanitary product having an attachment means, so that said pocket may be adhered to said sanitary product at the user's discretion.

4. The disposal system of claim 1 wherein said bag is laterally folded into its reduced configuration.

5. The disposal system of claim 1 wherein said bag has at least one longitudinal fold prior to being folded into its reduced configuration.

6. The disposal system of claim 1 wherein said bag is flexible so that it may be inverted during placement of the soiled sanitary product into said bag.

7. The disposal unit of claim 2 wherein said attachment means is a quick release contact adhesive.

8. The disposal unit of claim 2 further comprising a protective covering placed on top of said attachment means.

9. The disposal unit of claim 8 wherein a corner of said unit is without attachment means, so that said protective covering can be easily peeled off.

10. The disposal system of claim 1 further comprising a deodorizer disposed in said pocket.

11. The disposal system of claim 1 wherein said disposable sanitary product is an adult incontinence product.

12. The disposal system of claim 1 wherein said disposable sanitary product is a diaper.

13. The disposal system of claim 1 wherein said bag, said short tag, said long tag, and said pocket are all made out of one piece of continuous material.
